(19) United States
(12) Patent Application Publication
(10) Pub. No.: US 2014/0114208 A1
(43) Pub. Date: Apr. 24, 2014

(54) METHOD OF TREATING VISION DISORDERS

(71) Applicants: Craig Smith, Seattle, WA (US); Yi- Zhong Wang, Plano, TX (US); Michael B. Bartlett, Richardson, TX (US)

(72) Inventors: Craig Smith, Seattle, WA (US); Yi- Zhong Wang, Plano, TX (US); Michael B. Bartlett, Richardson, TX (US)

(73) Assignee: Novartis AG, Basel (CH)

(21) Appl. No.: 14/119,477

(22) PCT Filed: May 4, 2012
(86) PCT No.: PCT/US2012/036425

§ 371 Date: Nov. 22, 2013

Related U.S. Application Data
(60) Provisional application No. 61/490,774, filed on May 27, 2011.

(51) Int. Cl. A61B 5/00; A61K 38/17; A61B 5/117; A61B 3/10

(52) U.S. Cl. CPC A61B 5/4848; A61B 3/10; A61B 5/4839; A61B 5/6898; A61K 38/179; A61B 5/0022; A61B 5/1172; A61B 5/0077; A61B 5/749; A61B 5/742
USPC 600/558

(57) ABSTRACT

This invention is in the field of the treatment of eye disorders. In particular, it relates to the use of a remote monitoring system for determining patient response to therapeutic treatment, in particular with VEGF antagonists.

### TECHNICAL FIELD

This invention is in the field of the treatment of eye disorders. In particular, it relates to the use of a remote monitoring system for determining patient response to therapeutic treatments, in particular treatment with VEGF antagonists.

### BACKGROUND ART

Eye disorders mediated by VEGF such as age-related macular degeneration are a major public health problem that have a devastating effect upon patients and marked adverse financial consequences for economies. One study estimated that the cost of age-related macular degeneration to the US economy in terms of losses to the gross domestic product to be in the region of $30 billion (Brown et al. 2005, Trans Am Ophthalmol Soc. 103:173-186).

Of course, treatments for such disorders exist, including ranibizumab (Lucentis®), while others are currently in clinical trials, such as the VEGF Trap-Eye (aflibercept, EYLEA®) being developed by Regeneron and Bayer. Off label treatment using bevacizumab (Avastin®) has also been described.

Traditionally, the therapies are given according to strict (fixed) dosing regimens. However, there is evidence to suggest that in some cases treating “as needed” (or pro re nata) can result in the same or similar therapeutic outcome as treatment by such a strict dosing regimen. This still requires regular patient monitoring by a physician, which is both a burden on the physician's and patient's time.

There is therefore a need for a method of monitoring patient response to treatment of eye disorders which is convenient to both the patient and physician. There is a further need for a method of monitoring patient response to treatment of eye disorders and correlating such response to a therapeutic regime or protocol.

### DISCLOSURE OF THE INVENTION

It has been discovered that it is possible to monitor patient vision remotely using a hand held device which can measure the patient's visual function and then communicate the results to the physician (or other caregiver). In one embodiment, the hand held device may send the results remotely to the physician. The physician is then able to monitor the patient's response to treatment, perhaps even on a more frequent basis than would traditionally be feasible and then decide on if and when the patient's treatment should stop and if and when the patient should be retreated.

The invention provides a method of treating an eye disorder in a patient, wherein (i) the patient is administered a therapy, and (ii) the patient's response to treatment is monitored remotely by the physician.

The administration of the drug may be performed by the physician or caregiver, or be self-administered by the patient. The delivery route may be as approved for the therapy selected, such as subcutaneous injection, IV injection, intra-ocular injection, intra-vitreal injection, oral, inhalation, topical or other routes as known to the art. In one embodiment, the therapy may comprise non-drug therapy.

The invention further provides a method of treating an eye disorder in a patient, wherein (i) the patient is administered a VEGF antagonist, and (ii) the patient's response to treatment is monitored remotely by the physician.

The methods may further comprise the step of (iii) altering the patient's treatment regime such that visual function is maintained above a threshold level.

The methods may also further comprise the initial step of preliminarily assessing visual function prior to selecting a treatment.

In another embodiment, the invention provides a method of determining when a patient suffering from an eye disorder requires retreatment, comprising the steps of (i) measuring the patient's visual function, (ii) administering a therapy, such as a VEGF antagonist, (iii) monitoring the patient's visual function remotely, and (iv) retreating the patient when visual function drops below a threshold level. In this embodiment, step (ii) above may be modified such that the therapy (such as a VEGF antagonist) is administered at regular intervals until a stable level of visual function is maintained. Optionally, step (i) may be carried out remotely, but typically it is carried out in person by the physician in person. Optionally, between steps (iii) and (iv), the patient's visual function may be re-measured in person by a physician.

The invention also provides a VEGF antagonist for use in treating an eye disorder, wherein the patient's response to treatment is monitored remotely by the physician. Such a use may also comprise the step of altering the patient's treatment regime such that visual function is maintained above a threshold level.

In one embodiment, the monitoring is carried out using a portable device. In one embodiment, the monitoring is carried out using a non-portable device. In one embodiment, the monitoring is carried out using a hand held device.

In one embodiment the invention provides for a method of assessing, evaluating and/or treating a subject having a condition, disease or disorder which has a component which manifests in a visual test such as described herein. For example, a subject having a neurological condition, disease or disorder may be assessed, evaluated and/or treated with respect to such condition, disease or disorder, in accordance with embodiments described herein.

A “VEGF antagonist” refers to a molecule capable of neutralizing, blocking, inhibiting, abrogating, reducing or interfering with VEGF activities including its binding to one or more VEGF receptors. VEGF antagonists include anti-VEGF antibodies and antigen-binding fragments thereof, receptor molecules and derivatives which bind specifically to VEGF thereby sequestering its binding to one or more receptors, anti-VEGF receptor antibodies and VEGF receptor antagonists such as small molecule inhibitors of the VEGFR tyrosine kinases, and fusions proteins. In one embodiment, the VEGF antagonist is an antibody. In one embodiment, the VEGF antagonist is a mimetic of the VEGF receptor. In one embodiment, the VEGF antagonist is ranibizumab. In one embodiment, ranibizumab is administered in a dose of 0.3 mg or 0.5 mg. In another embodiment, the VEGF antagonist is VEGF Trap-Eye (aflibercept, EYLEA®). In one embodiment, VEGF Trap-Eye is administered in a dose of 0.5 mg or 2 mg. In one embodiment, the VEGF antagonist is bevacizumab (Avastin®). In one embodiment, bevacizumab is administered in a dose of 1.25 mg or 2.5 mg.

In one embodiment, the eye disorder is selected from choroidal neovascularisation, age-related macular degeneration (both wet and dry forms), macular edema secondary to retinal vein occlusion (RVO) including both branch RVO (bRVO) and central RVO (cRVO), choroidal neovascularisation secondary to pathologic myopia (PM), or diabetic macular edema (DME). In one embodiment, the eye disorder is wet age-related macular degeneration (wet AMD).

As the patient's response to treatment is remotely monitored, the physician can easily determine when the patient should stop treatment and when they should return for re-treatment. Treatment would normally continue until the patient's visual function ceases to show improvement. Re-treatment would normally occur when the patient's visual function begins to deteriorate, or deteriorates at a pre-defined rate or beyond a certain threshold.

Thus, the physician can modify the treatment regimen that the patient receives in order to create a regimen specifically tailored to the patient, to offer maximum benefit to the patient (e.g. in terms of minimal number of treatment procedures and reduced likelihood of adverse events as the patient is only treated when needed), physician (e.g. in terms of the patient is only seen when needed, thus potentially freeing up physician time to see other patients) and payor (e.g. in that the patient only receives the number of treatments required to maintain eyesight/treat the disorder and is not given extra unnecessary costly treatments). Such personalised medicine and frequent monitoring is also believed to result in a better patient outcome, such as reduced deterioration of visual function, decreased likelihood of adverse events and improved satisfaction.

### Remote Monitoring

By remote monitoring, we mean that the patient's response to treatment (in terms of improved visual function) is monitored by the physician without seeing the patient in person. Thus, the patient may be able to measure his own response to treatment and submit the results to the physician for evaluation.

One way of doing this may be via a remote device that is able to carry out a sight test and automatically supply the results to the physician. In one embodiment, such a device is a hand held device, such as a personal digital assistant (PDA), gaming console (e.g. Nintendo DS™) tablet computing device (e,g. an iPad™) or smart phone (e.g. an iPhone™). Of course, the device may be one specifically manufactured for the task. Examples of such devices for testing vision are found in WO2010/132304 and WO2010/132305, the contents of which are incorporated by reference. Other suitable devices which can serve as a platform for the sight test comprise personal computers, laptops, desktops, notepads, mainframes, or other devices with sufficient processing power and display capabilities.

Typically the device will have a display, cursor control and an interface port. The device may further comprise a camera. Thus, the device will display images to the patient who can then provide input via the device. Preferably the display is a touch-screen, such that the patient can input directly on the screen.

In one embodiment the display meets one or more of the following standards: (a) ANSI 280.21-1992 (R2004) for background luminance (i.e. it falls within the range 80-320 Cd/m²), (b) a contrast ratio of 300:1, 600:1 or greater, in accordance with ISO 8596, and (c) ISO 8596:1994(E) (i.e. has a colour temperature of 2500K to 7000K).

In one embodiment, the device comprises a camera that faces the patent while the test is being completed. The device may have facial recognition software loaded that, in combination with the camera, (a) allows the device to confirm the identity of the patient completing the test, (b) allows the device to confirm that the correct eye is being tested (i.e. that the patient has closed the correct eye, or has covered the correct eye with a patch), (c) allows the device to confirm the ambient light level/luminance in the location where the device and patient are located and/or (d) allows the device to confirm that the screen on which the test is displayed is maintained at a constant, preset distance from the patient's eyes. If any one or more of the following conditions are satisfied: (a) it is not possible to confirm the identity of the patient, (b) the incorrect eye is closed/covered, (c) the ambient light level/luminance is above or below predetermined threshold levels (e.g. 120 cd/m²+20%) and (d) the screen is too close or too far away from the patient's eyes, then the device will display a warning to the patient and optionally also send an alert to the physician. Optionally, the physician may also receive an alert if the patient receives one or more such warnings (e.g. 3, 5, 7, 10 or more such warnings). The device may additionally or alternatively include appropriate hardware or software to enable other biometrics for determining patient identity, such as fingerprint or retinal pattern scan.

In one embodiment, the device may measure the distance between the patient's eyes and the device and adjust the test accordingly. Thus, if the device is positioned further away from the patient, the size of the letters/figures used in the test may be increased. Conversely, if the device is positioned closer to the patient, the size of the letters/figures used in the test may be decreased. Distance measuring may be implemented by non contact sensors, for example, by the use of ultrasonic or infrared sensors.

In one embodiment, the patient may wear an eye patch over the eye that is not being tested. Such an eye patch may comprise a shape or figure that is recognised by the device such that the distance from the device to the patient's eyes can be measured more accurately.

In one embodiment, the device may further comprise a microphone, a speaker and voice recognition software. Thus the device could be operated by the patient using voice commands.

Various types of sight test for measuring visual function may be used, such as the amsler grid test, snellen acuity chart, “tumbling E” chart, “Landolt C” chart, moving line test, crosshair alignment pattern etc., many of which are described in U.S. 2007/0200927. However, it is preferred to use the dynamic shape discrimination vision test described in U.S. 2009/0273758 (incorporated by reference), also known as the shape discrimination hyperacuity (SDH) test. The SDH test is designed to bypass suppressive brain mechanisms by using a forced-choice paradigm and to employ a sensitive global discrimination hyperacuity function to detect central visual distortion associated with various forms of retinal disorders. It is easy to learn and operate and has been designed to keep false positive test results to a minimum. The patient may be requested to complete two or more such types of tests consecutively, in order to give a more accurate readout of visual acuity. Thus, in one embodiment, the patient may be requested to complete the SDH test as well as a test based on the snellen acuity chart.

In one embodiment, the remote device comprises a touch-based graphic user-interface (GUI), a visual stimulus generator, a psychophysical procedure, and a threshold-estimating algorithm. The GUI allows the patient to input information and guides the patient through the test. The visual stimulus generator creates various circular contour shapes used in the SDH test. The psychophysical procedure is a forced-choice, adaptive method that determines stimulus levels to be used at each test trial based on the patient's response. The threshold-estimating algorithm is used to obtain measurements of shape discrimination hyperacuity from psychophysical data. In one embodiment, the device is loaded with myVisionTrack™ software.

Thus, as frequently as requested by the physician, the patient can take the sight test. The results of this test can then be sent by the patient to the physician. The submission may be via a variety of pathways, protocols and formats, such as a realtime uplink from the monitoring device, a store-and-forward protocol, an indirect upload or link, a reduction to tangible form and manual delivery or the like. In some embodiments, the treatment may be adjusted without physician or caregiver intervention, as by a predetermined algorithm. In one embodiment, the results are sent automatically to the physician following completion of the test. In one embodiment, the results are sent “realtime” to the physician.

The patient may take the sight test about monthly, about every three weeks, about every two weeks, about every week, about every three days, about every day or more frequently. The physician will be able to determine with the patient the appropriate frequency. In one embodiment, the sight test is taken daily. The frequency that the sight test is taken may be varied. Thus, directly following treatment, the sight test may be taken more frequently (e.g. daily) and after two weeks, the sight test may be taken less frequently (e.g. every 3 days) and vice versa. The physician can communicate to the patient through the device any such changes in frequency.

The physician may also, via the device, be able to schedule an appointment with the patient for the next treatment. In the case of therapies or drugs which can be self-administered, the device may schedule such administration, and alert the patient as needed. In the case of emergencies, such as when the patients visual function score significantly decreases, e.g. potentially due to an adverse event, the device may be able to automatically alert the physician and make an emergency appointment for the patient. The patient may also be able to report adverse events and serious adverse events to the physician using the device. Such reporting may be via a short series of questions asked by the device following the sight test.

### Response To Treatment And Dosing

Using the testing method described above, the physician can develop a profile of the patient's response to treatment. Thus, it will be possible for the physician to profile any improvement in a patient's visual function following treatment and conversely any decline in visual function. Thus, while a standard dosing regime for a given drug may be e.g. monthly, if the profile shows that the patient's visual function is not declining, then the physician may choose to delay further treatment until such a decline is evident. This reduces the number of treatments a patient receives, saving both time and money. Conversely, if the sight test indicates visual function is declining at a faster than expected rate, therapeutic interventions may be more frequent. Visual function testing may also be conducted more frequently in such cases. The device itself can be used to alert the patient to the need to conduct the test.

In one embodiment, the patient is treated at regular intervals until no further improvement in visual function is seen following two or more (i.e. 2, 3, 4, 5 or more) consecutive treatments. In another embodiment, the patient is treated at regular intervals until they achieve a best corrected visual acuity (BCVA) score of 80 or more (i.e. 81, 82, 83, 84, 85, 86, 87, 88, 89, 90 or more) following two or more (i.e. 2, 3, 4, 5 or more) consecutive treatments. In one embodiment, the threshold BCVA score is 84. In another embodiment, the patient is treated at regular intervals until no further improvement is seen following two or more (i.e. 2, 3, 4, 5 or more) consecutive treatments, as determined by the SDH test score.

In such a case, the regular intervals between treatments may be about one week, two weeks, one month, six weeks, two months or longer. For example, ranibizumab is typically administered monthly, while the VEGF Trap-Eye (aflibercept, EYLEA®) is typically administered every two months (after 3 monthly loading doses). Thus, assuming a monthly dosing regime, if the patient's visual function improves following treatment at month 0, 1, 2, 3, 4 and then stabilises and shows no further improvement following treatment at months 5 and 6, no further treatment would be given. Of course, the patient's visual function would still be monitored using the device. However, once a patient's visual function starts to decline beyond a pre-set threshold, treatment would resume.

In one embodiment, further treatment is given only when the patient's visual function declines by about 1%, 2%, 3%, 5%, 10% or more from a baseline level. For example, if following treatment the patient gets the SDH test correct 20/25 times for 5 weeks, but then after 6 or 7 weeks only gets the SDH test correct 15/25 times, the physician will know that the patient's visual function is decreasing, requiring re-treatment. In one embodiment, said baseline level is the stable level achieved causing the physician to stop treatment.

In another embodiment, retreatment is given when a patient's score (i.e. number of correct answers) in two or more (i.e. 2, 3, 4, 5, 7, 10 or more) consecutive tests decreases by x%, compared to the average score over the preceding y days. In such a case the test used is one where there is a simple right or wrong answer, such as the SDH test, “tumbling E” chart or “Landolt C” chart. In one embodiment, x is 1%, 2%, 3%, 5%, 10% or more. In one embodiment, y is 3, 5, 7, 10, 12, 14, 15, 21, 30, 45, 60 days or more.

In the above, when we refer to a patient receiving treatment, we mean single administration of the therapeutic agent (e.g. ranibizumab, aflibercept) at the appropriate dosage as determined by their physician.

It may be desirable for the patient to still be examined by the physician at regular intervals. Indeed this is important when the patient first starts using the device to ensure that it is properly calibrated and the patient can use it effectively. Thus, in one embodiment, the patient undergoes examination by the physician about every two weeks, about every month, about every two months, about every three months or less frequently.

### Kits

In one embodiment, the invention provides a kit comprising the remote device, the vision testing software and instructions for use. The kit may further optionally provide a therapeutic agent (e.g. a VEGF antagonist). If a kit is intended for the patient to self-administer therapy, the kit may comprise all these parts. Alternatively, a kit intended for the physician may comprise two main parts, the first part comprising the therapeutic agent (optionally further including instructions, and/or a delivery device, such as a syringe), the second part comprising the remote device, the vision testing software and optionally instructions for use (said second part intended for the patient). The vision testing software may be pre-loaded onto the remote device.

### General

The term “comprising” means “including” as well as “consisting” e.g. a composition “comprising” X may consist exclusively of X or may include something additional e.g. X+Y.

The term “about” in relation to a numerical value x means, for example, x±10%.

### NUMBERED EMBODIMENTS OF THE INVENTION

1. A method of treating an eye disorder in a patient, wherein (i) the patient is administered a therapy, and (ii) the patient's response to treatment is monitored remotely by the physician.

2. The method of embodiment 1, further comprising the step of (iii) altering the patient's treatment regime such that visual function is maintained.

3. A method of determining when a patient suffering from an eye disorder requires retreatment, comprising the steps of (i) measuring the patient's visual function, (ii) administering a therapy, (iii) monitoring the patient's visual function remotely, and (iv) retreating the patient when visual function drops below a threshold level.

4. The method of any one of embodiments 1-3, wherein the patient is administered a VEGF antagonist.

5. A VEGF antagonist for use in treating an eye disorder, wherein the patient's response to treatment is monitored remotely by the physician.

6. The use according to embodiment 5, further comprising the step of altering the patient's treatment regime such that visual function is maintained above a threshold level.

7. The method according to any of embodiments 1-4 or use according to embodiment 5 or embodiment 6 wherein the eye disorder being treated is selected from: choroidal neovascularisation, age-related macular degeneration (both wet and dry forms), macular edema secondary to retinal vein occlusion (RVO) including both branch RVO (bRVO) and central RVO (cRVO), choroidal neovascularisation secondary to pathologic myopia (PM), or diabetic macular edema (DME).

8. The method or use according to any previous embodiment, wherein the patient's response to treatment is measured using a remote device that is able to carry out a sight test and supply the results to the physician.

9. The method or use according to any previous embodiment, wherein the remote device is hand held.

10. The method or use according to embodiment 9, wherein the hand held device is a PDA, gaming console or smart phone.

11. The method or use according to any previous embodiment, wherein the sight test is the dynamic shape discrimination vision test described in U.S. 2009/0273758.

12. The method or use according to any previous embodiment, wherein the results of the test are sent realtime to the physician.

13. The method or use according to any previous embodiment, wherein the patient is treated with ranibizumab, bevacizumab or VEGF Trap-Eye (aflibercept).

14. The method or use according to any of embodiments 7-13, wherein the sight test is the amsler grid test, snellen acuity chart, “tumbling E” chart, “Landolt C” chart, moving line test, crosshair alignment pattern test or the SDH test.

15. The method or use according to embodiment 14, wherein the sight test is the SDH test, “tumbling E” chart or “Landolt C” chart.

16. The method or use according to embodiment 15, wherein treatment is administered until no further improvement in visual function is seen following two or more (i.e. 2, 3, 4, 5 or more) consecutive treatments.

17. The method or use according to embodiment 15, wherein treatment is administered until the patient achieves a best corrected visual acuity (BCVA) score of 80 or more (i.e. 81, 82, 83, 84, 85, 86, 87, 88, 89, 90 or more) following two or more (i.e. 2, 3, 4, 5 or more) consecutive treatments.

18. The method or use according to embodiment 15, wherein treatment is administered until no further improvement is seen following two or more (i.e. 2, 3, 4, 5 or more) consecutive treatments, as determined by the SDH test score.

19. The method or use according to any of embodiments 15-18, wherein retreatment is given when the patient's score (i.e. number of correct answers) in two or more (i.e. 2, 3, 4, 5, 7, 10 or more) consecutive tests decreases by x%, compared to the average score over the preceding y days, wherein x is 1%, 2%, 3%, 5%, 10% or more, and y is 3, 5, 7, 10, 12, 14, 15, 21 days or more.

20. The method or use according to any of embodiments 15-18, wherein retreatment is given when the patient's visual function declines by 1%, 2%, 3%, 5%, 10% or more from a baseline level.

21. The method or use according to embodiment 20, wherein said baseline level is the stable level achieved causing the cessation of treatment.

22. The method or use according to any previous embodiment, wherein the VEGF antagonist is (a) ranibizumab administered at a dose of 0.5 mg, or (b) aflibercept administered at a dose of 2 mg.

23. The method or use according to any previous embodiment, wherein the patient completes the dynamic shape discrimination vision test daily and is examined by the physician monthly.

24. The method or use according to any previous embodiment, wherein patient self-administers the therapy and the device instructs when the therapy should be administered according to a predetermined algorithm.

25. The method or use according to any previous embodiment, wherein following a significant decrease in visual function as determined by the device, the physician is automatically alerted and an emergency appointment for the patient to see the physician is made.

26. The method or use according to any previous embodiment, wherein the patient consecutively completes two or more types of vision test disclosed in embodiment 14.

27. A kit comprising a remote device, vision testing software and instructions for use.

28. The kit of embodiment 27, further comprising a therapeutic agent.

29. The kit of embodiment 28, wherein said therapeutic agent is a VEGF antagonist.

30. The kit of any of embodiments 27-29, wherein said kit further comprises a delivery device and instructions for use.

### MODES FOR CARRYING OUT THE INVENTION

### Clinical Trial 1

Approximately 160 patients suffering from choroidal neovascularisation secondary to age-related macular degeneration in at least one eye are enrolled into the trial. These may include patients who have previously been treated with ranibizumab or another anti-VEGF therapy.

All eyes affected with CNV secondary to AMD at the time of entering the study are analyzed as study eyes. Healthy eyes are also evaluated to allow for differentiation against AMD eyes. At the first visit, the patient is shown how to use the device and takes the first test (SDH test) using the device loaded with myVisionTrack™ software. If the physician is not confident in the patient's ability to use the device outside of the clinic, the screening period is extended to a maximum of 7 days so that the patient has the opportunity to familiarize themselves with the device. Thereafter, the patient is asked to take the test daily for each eye for a period of 16 weeks at around the same time of day.

During the 16-weeks study period, patients undergo clinical assessments by the physician every 4 weeks, including checking best corrected visual acuity (BCVA) with an ETDRS (Early Treatment Diabetic Retinopathy Study) chart, and evaluations of anatomic features of the retina and choroid (such as optical coherence tomography (OCT), ophthalmoscopy, etc).

Ranibizumab treatment is continued or started at the physician's discretion.

Of the 160 patients (mean age 76.6 years) who began the trial, at 24 centres across the US, 147 completed the 16-week trial. 92.5% of the patients confirmed that the device loaded with myVisionTrack™ software was easy to use. The data suggest some correlation between mVT assessment values and BCVA values as determined by the physicians. Such a system has the potential to measure clinically meaningful change in neovascular AMD.

It will be understood that the invention has been described by way of example only and modifications may be made whilst remaining within the scope and spirit of the invention.

1. A method of treating an eye disorder in a patient, comprising (i) treating the patient with a therapy, and (ii) remotely monitoring the patient's response to treatment using a remote device that is capable of carrying out a sight test and supplying results of said sight test to an individual monitoring the patient.

2. The method of claim 1, further comprising the step of (iii) altering the patient's treatment regime such that visual function is maintained.

3. A method of determining when a patient suffering from an eye disorder requires retreatment, comprising the steps of (i) measuring the patient's visual function, (ii) administering a therapy, (iii) monitoring the patient's visual function remotely using a device that is capable of carrying out a sight test and supplying results of said sight test to an individual monitoring the patient, and (iv) retreating the patient when visual function drops below a threshold level.

4. The method of claim 1, wherein the patient is administered a VEGF antagonist.

5. (canceled)

6. The method of claim 1, further comprising the step of altering the patient's treatment regime such that visual function is maintained above a threshold level.

7. The method according to claim 1 wherein the eye disorder being treated is selected from: choroidal neovascularisation, age-related macular degeneration (both wet and dry forms), macular edema secondary to retinal vein occlusion (RVO) including both branch RVO (bRVO) and central RVO (cRVO), choroidal neovascularisation secondary to pathologic myopia (PM), or diabetic macular edema (DME).

8. (canceled)

9. The method according to claim 7, wherein the remote device is hand held.

10. The method according to claim 9, wherein the hand held device is a PDA, gaming console or smart phone.

11. The method according to claim 1, wherein the sight test is a dynamic shape discrimination vision test.

12. The method of claim 1, wherein the results of the test are sent realtime to the individual.

13. The method of claim 4, wherein the VEGF antagonist is selected from the group consisting of ranibizumab, bevacizumab and VEGF Trap-Eye (aflibercept).

14. The method of claim 1, wherein the sight test is the amsler grid test, snellen acuity chart, “tumbling E” chart, “Landolt C” chart, moving line test, crosshair alignment pattern test or the SDH test.

15. The method of claim 14, wherein the sight test is the SDH test, “tumbling E” chart or “Landolt C” chart.

16. The method according to claim 15, wherein treatment is administered until no further improvement in visual function is seen following two or more consecutive treatments.

17. The method or use according to claim 15, wherein treatment is administered until the patient achieves a best corrected visual acuity (BCVA) score of 80 or more following two or more consecutive treatments.

18. The method according to claim 15, wherein treatment is administered until no further improvement is seen following two or more consecutive treatments, as determined by the SDH test score.

19. The method according to claim 3, wherein retreatment is given when the patient's score in two or more consecutive tests decreases by x%, compared to the average score over the preceding y days, wherein x is 1%, 2%, 3%, 5%, 10% or more, and y is 3, 5, 7, 10, 12, 14, 15, 21 days or more.

20. The method according to claim 3, wherein retreatment is given when the patient's visual function declines by 1%, 2%, 3%, 5%, 10% or more from a baseline level.

21. The method according to claim 20, wherein said baseline level is the stable level achieved causing the cessation of treatment.

22. The method according to claim 13, wherein the VEGF antagonist is (a) ranibizumab administered at a dose of 0.5 mg, or (b) aflibercept administered at a dose of 2 mg.

23. The method or use according to any previous, claim 11, wherein the patient completes the dynamic shape discrimination vision test daily.

24. The method according to claim 1, wherein the device instructs when the therapy should be administered according to a pre-determined algorithm.

25. The method according to claim 1, wherein following a significant decrease in visual function as determined by the device, the physician is automatically alerted and an emergency appointment for the patient to see the physician is made.

26. The method according to claim 14, wherein the patient consecutively completes two or more of the amsler grid test, snellen acuity chart, “tumbling E” chart, “Landolt C” chart, moving line test, crosshair alignment pattern test or the SDH test.

27. A kit comprising a remote device, vision testing software and instructions for use in the method of claim 1.

28. The kit of claim 27, further comprising a therapeutic agent.

29. The kit of claim 28, wherein said therapeutic agent is a VEGF antagonist.

30. The kit of claim 27, wherein said kit further comprises a delivery device and instructions for use.
